(19) United States
(12) Patent Application Publication
(10) Pub. No.: US 2019/0204402 A1
(43) Pub. Date: Jul. 4, 2019

(54) METHOD FOR POST-PROCESSING MRI IMAGES TO OBTAIN HEPATIC PERFUSION AND TRANSPORT PARAMETERS

(71) Applicants: INSERM (INSTITUT NATIONAL DE LA SANTE DE LA RECHERCHE MEDICALE), Paris (FR); UNIVERSITE PARIS DIDEROT - PARIS 7, Paris (FR); CENTRE NATIONAL DE LA RECHERCHE SCIENTIFIQUE, Paris (FR); ASSISTANCE PUBLIQUE - HOPITAUX DE PARIS, Paris (FR)

(72) Inventors: Benjamin LEPORQ, Villeurbanne Cedex (FR); Jean-Luc DAIRE, Paris (FR); Bernard VAN BEERS, Paris (FR)

(21) Appl. No.: 16/331,732

(22) PCT Filed: Sep. 13, 2017
(86) PCT No.: PCT/EP2017/072974

§ 371 Date: Mar. 8, 2019

(30) Foreign Application Priority Data
Sep. 13, 2016 (EP) 16306156.7

(51) Int. Cl. G01R 33/563; A61B 5/055; G01R 33/56; A61B 5/026; A61B 5/0275; A61B 5/00

(52) U.S. Cl. CPC G01R 33/56366; A61B 5/055; G01R 33/5601; G01R 33/5635; A61B 5/113; A61B 5/0275; A61B 5/4244; G06T 2207/30056; A61B 5/7207; A61B 5/0263

(57) ABSTRACT

The invention relates to liver diseases. Liver diseases notably encompass chronic liver disease and liver cancer (a liver primitive cancer or metastasis). There is therefore a need to be able to extract biomarkers for subjects to suffer from this disease. As a consequence, the inventors worked on a method for post-processing images of a region of interest to obtain at least one perfusion parameter and at least one transport parameter. Such method enables to obtain a method which can be implemented on computer and provides access to relevant parameters for liver diseases in an easier and more accurate way. This method may be applied for predicting that a subject is at risk of suffering from such disease, diagnosing a disease, identifying a therapeutic or a biomarker and screening compounds useful as a medicine.

### TECHNICAL FIELD OF THE INVENTION

The present invention concerns a method for post-processing images of a region of interest of a subject. The invention concerns a method for predicting that a subject is at risk of suffering from a liver disease. The invention also relates to a method for diagnosing a liver disease. The invention also concerns a method for identifying a therapeutic target for preventing and/or treating a liver disease. The invention also relates to a method for identifying a biomarker, the biomarker being a diagnostic biomarker of a liver disease, a susceptibility biomarker of a liver disease, a prognostic biomarker of a liver disease or a predictive biomarker in response to the treatment of a liver disease. The invention also concerns a method for screening a compound useful as a medicine, the compound having an effect on a known therapeutical target, for preventing and/or treating a liver disease. The invention also relates to the associated computer program products and a computer readable medium.

### BACKGROUND OF THE INVENTION

Liver diseases concern a high number of people in the world.

Liver diseases notably encompass chronic liver disease and liver cancer (a liver primitive cancer or metastasis)

There is therefore a need to be able to extract biomarkers for subjects to suffer from this disease.

The main issue comes from a double choice: the choice of the properties to be measured and the choice of the techniques to be used for measuring the properties.

For instance, for liver disease, it is desirable to obtain information on the tissue cellularity, their perfusion, the degree of molecular transport in hepatocytes and their visco-elasticity.

As an example, the tissue perfusion may be investigated by using dynamic contrast-enhanced MRI, or perfusion computed tomography and the transport function of the hepatocytes may be measured by using gadoxetate MRI enhancement analyses or blood tests such as aminotransferase, bilirubin or gamma glutamyl transferase levels.

However, some of these techniques are not compatible together in so far as they require two different experiments.

Furthermore, these techniques do not provide in combination a good accuracy in the prediction of the risk for a subject to suffer from disease liver disease.

### SUMMARY OF THE INVENTION

The invention aims at providing a method which can be achieved in only one experiment and provides the best accuracy in the prediction of the risk for a subject to suffer from a liver disease.

By liver disease, it is meant a disease that affects the liver.

According to an embodiment, the liver disease is one of a chronic liver disease, a liver cancer.

Chronic liver disease in the clinical context is a disease process of the liver that involves a process of progressive destruction and regeneration of the liver parenchyma leading to fibrosis and cirrhosis. Chronic liver disease refers to disease of the liver which lasts over a period of six months. It consists of a wide range of liver pathologies which include inflammation (chronic hepatitis), liver cirrhosis, and hepatocellular carcinoma.

Liver cancer, also known as hepatic cancer, is a cancer that originates in the liver. Liver tumors are discovered on medical imaging equipment or present themselves symptomatically as an abdominal mass, abdominal pain, yellow skin, nausea or liver dysfunction. The leading cause of liver cancer is cirrhosis due to either hepatitis B, hepatitis C, or alcohol.

According to an embodiment, the liver cancer is a liver primitive cancer or a liver metastasis.

To this end, the specification describes a method for post-processing images of a region of interest in a subject to obtain determined parameters, the determined parameters comprising at least one perfusion parameter and at least one transport parameter, the perfusion parameters being relative to the hepatic perfusion and the transport parameter being relative to the hepatic function transport, the images being acquired with a magnetic resonance imaging technique, the magnetic resonance imaging technique being enhanced by a contrast agent, the magnetic resonance imaging technique involving successive echoes of a multiple-gradient echo sequence, each image associating to each pixel of the image the amplitude of the measured signal in the magnetic resonance imaging technique and the phase of the measured signal in the magnetic resonance imaging technique. The method for post-processing comprising at least the phase of extracting a time intensity-curve or signal intensity according to the time for at least one pixel of the images, to obtain at least one time-intensity curve, the phase of converting the time-intensity curves in a concentration signal, a concentration signal being a signal representative of the evolution of the contrast agent concentration with time, the phase of calculating the at least one perfusion parameter and the at least one transport parameter by using a fitting procedure applied on a model, the model being a function which associates to a plurality of parameters each concentration signal, the plurality of parameters being parameters which characterizes the kinetics of the elimination of the contrast agent by the liver, the liver being represented as a three-compartment organ with an extracellular compartment, a hepatocyte compartment and the intra-hepatic bile ducts, the plurality of parameters comprising at least one perfusion parameter and at least one transport parameter. The fitting procedure is applied in two steps: a first step during which several parameters of the model are set to zero, the model becoming a simplified model corresponding to the liver being represented as a two-compartment organ with an extracellular compartment and a hepatocyte compartment only, to obtain determined parameters with a determined value and a second step during which several parameters are set the determined value, to obtain the at least one perfusion parameter and the at least one transport parameter. Each step is achieved with a fitting technique being a non-linear least-square fitting technique using pseudo-random initial conditions.

Such fitting technique enables to obtain more accurate results than the method described in the article from Celine Giraudeau et al. whose title is “Gadoxetate-enhanced MRI in rats with liver cirrhosis: comparison between functional liver parameters obtained with deconvolution analysis and compartmental models as markers of hepatocyte transporter expression” Proceedings of the International Society for Magnetic Resonance in Medicine, vol. 22, Apr. 25, 2014.

This technique includes two inputs in the extracellular compartment of the quantification model rather only one in the article from C. Giraudeau et al. Therefore, the technique presented in this application is more “physiologic” since the liver has two vascular supplies (arterial and portal). In addition, rather than deconvolution-based approaches such as used in the Giraudeau et al method, the described technique used a dedicated data fitting procedure and allow to measure perfusion parameters. These latter are also known to be clinically relevant to assess liver fibrosis severity.

According to further aspects of the invention which are advantageous but not compulsory, the method for post-processing images might incorporate one or several of the following features, taken in any technically admissible combination:
the first step is applied on images during a first interval of time and the second step is applied on images during a second interval of time, the second interval of time including the first interval of time.
the ratio of the first interval of time to the second interval of time is inferior to 25%.
the first interval of time is comprises between 5 minutes to 10 minutes.
the phase of converting comprises a step of converting the concentration of contrast agent concentration in blood into the concentration of contrast agent concentration in plasma.
the phase of converting comprises a step of interpolating the concentration signal.
the plurality of parameters comprises the rate of exchanges between each of the three compartments.
the calculating phase comprises a step of calculating at least one of the hepatocyte uptake fraction, the extracellular mean transit time and the extracellular volume based on the determined at least one perfusion parameter and the at least one transport parameter, the calculated parameter being one of determined parameter.
the region of interest includes a part of the liver.

The specification describes a method for predicting that a subject is at risk of suffering from a liver disease, the method for predicting at least comprising the step of carrying out the steps of a method for post-processing images of the subject, to obtain determined parameters and the step of predicting that the subject is at risk of suffering from the liver disease based on the determined parameters.

Another application of such method for predicting is notably to evaluate the risk of postoperative liver failure after major liver resection. The postoperative liver failure after major liver resection is considered to be a liver disease in this context.

The specification also relates to a method for diagnosing a liver disease, the method for diagnosing at least comprising the step of carrying out the steps of a method for post-processing images of the subject, to obtain determined parameters and the step of diagnosing the liver disease based on the determined parameters.

The specification also concerns a method for identifying a therapeutic target for preventing and/or treating a liver disease, the method comprising the step of carrying out the steps of a method for post-processing images of a first subject, to obtain first determined parameters corresponding to the determined parameters for the first subject, the first subject being a subject suffering from the liver disease. The method for identifying further comprising the step of carrying out the steps of the method for post-processing images of a second subject, to obtain second determined parameters corresponding to the determined parameters for the second subject, the second subject being a subject not suffering from the liver disease, and the step of selecting a therapeutic target based on the comparison of the first and second determined parameters.

The specification also relates to a method for identifying a biomarker, the biomarker being a diagnostic biomarker of a liver disease, a susceptibility biomarker of a liver disease, a prognostic biomarker of a liver disease or a predictive biomarker in response to the treatment of a liver disease, the method comprising the step of carrying out the steps of a method for post-processing images of a first subject, to obtain first determined parameters, the first determined parameters corresponding to the determined parameters for the first subject, the first subject being a subject suffering from the liver disease. The method for identifying further comprising the step of carrying out the steps of the method for post-processing images of a second subject, to obtain second determined parameters corresponding to the determined parameters for the second subject, the second subject being a subject not suffering from the liver disease, and the step of selecting a biomarker based on the comparison of the first and second determined parameters.

The specification also concerns a method for screening a compound useful as a medicine, the compound having an effect on a known therapeutical target, for preventing and/or treating a liver disease, the method comprising a step of carrying out the steps of a method for post-processing images of a first subject, to obtain first determined parameters, the first determined parameters corresponding to the determined parameters for the first subject, the first subject being a subject suffering from the liver disease and having received the compound. The method also comprises a step of carrying out the steps of the method for post-processing images of a second subject, to obtain second determined parameters, the second determined parameters corresponding to the determined parameters for the second subject, the second subject being a subject suffering from the liver disease and not having received the compound. The method also comprises a step of selecting a compound based on the comparison of the first and second determined parameters.

The specification also describes a computer program product comprising instructions for carrying out the steps of any method chosen among the previously described method for post-processing, method for predicting, method for diagnosing, method for identifying a therapeutic target, method for identifying a biomarker and the method for screening a compound useful as a medicine when said computer program product is executed on a suitable computer device.

The specification also describes a computer readable medium having encoded

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 shows schematically a system and a computer program product whose interaction enables to carry out a method for post-processing images;

FIG. 2 shows a flowchart of the method for post-processing images;

FIG. 3 shows a schematic view of a pharmacokinetic model used in the method for post-processing images, and

FIGS. 4 to 17 illustrate the results obtained by an example of experiment corresponding to the carrying out of the method for post-processing.

### DETAILED DESCRIPTION OF SOME EMBODIMENTS

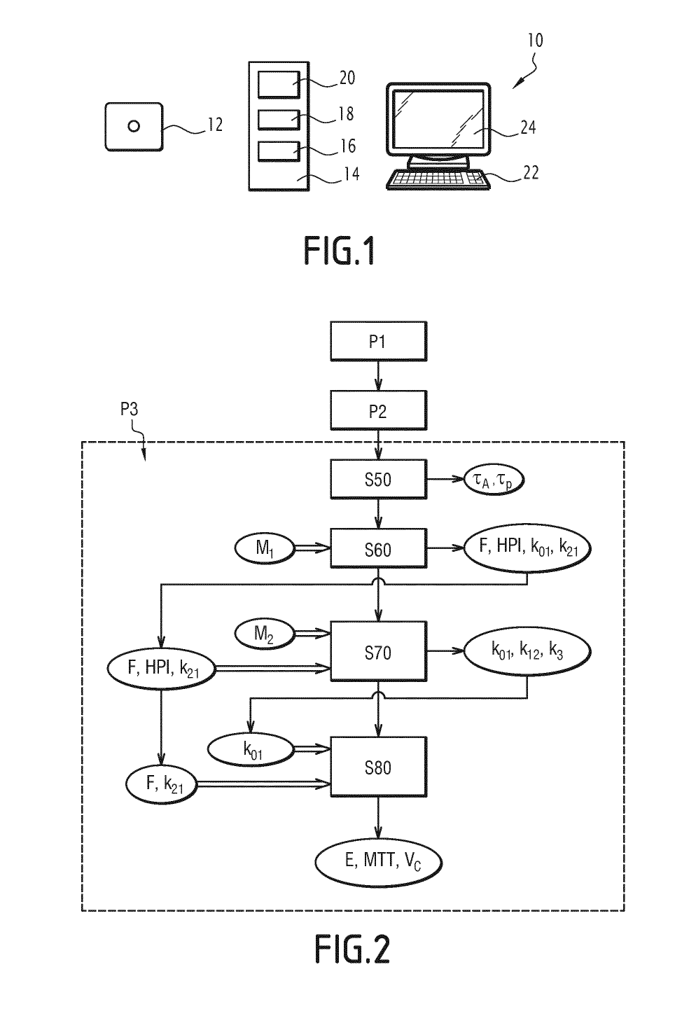

A system 10 and a computer program product 12 are represented in FIG. 1. The interaction between the computer program product 12 and the system 10 enables to carry out a method for post-processing images.

System 10 is a computer. In the present case, system 10 is a laptop.

More generally, system 10 is a computer or computing system, or similar electronic computing device adapted to manipulate and/or transform data represented as physical, such as electronic, quantities within the computing system's registers and/or memories into other data similarly represented as physical quantities within the computing system's memories, registers or other such information storage, transmission or display devices.

System 10 comprises a processor 14, a keyboard 22 and a display unit 24.

The processor 14 comprises a data-processing unit 16, memories 18 and a reader 20. The reader 20 is adapted to read a computer readable medium.

The computer program product 12 comprises a computer readable medium.

The computer readable medium is a medium that can be read by the reader of the processor. The computer readable medium is a medium suitable for storing electronic instructions, and capable of being coupled to a computer system bus.

Such computer readable storage medium is, for instance, a disk, a floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

A computer program is stored in the computer readable storage medium. The computer program comprises one or more stored sequence of program instructions.

The computer program is loadable into the data-processing unit and adapted to cause execution of the method for post-processing images when the computer program is run by the data-processing unit.

Operation of the system 10 is now described by illustrating an example of carrying out the method for post-processing images as illustrated by the flowchart of FIG. 2.

The images post-processed in the method for post-processing images are images of a region of interest in a subject.

The region of interest includes a part of the liver.

The subject is usually human beings.

The subject can also be animals, such as mice, rats, rabbits or primates.

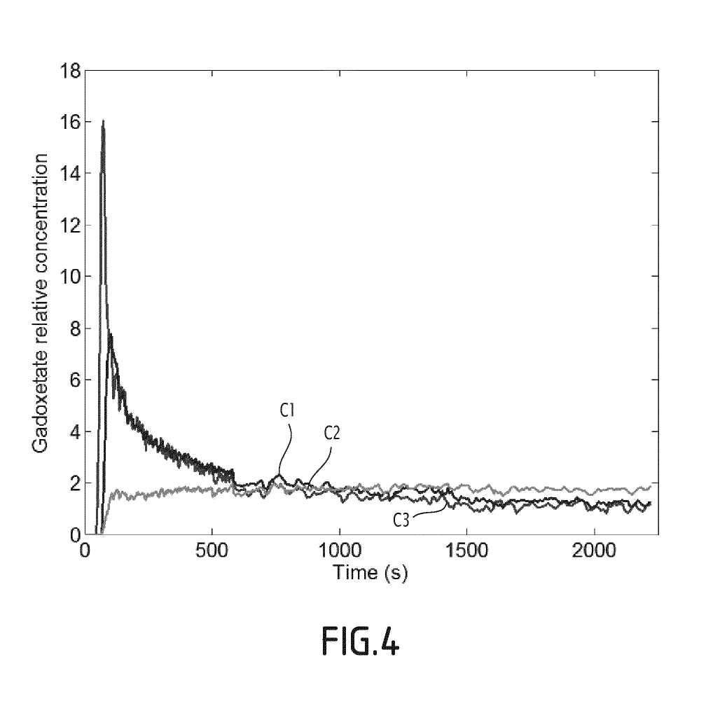

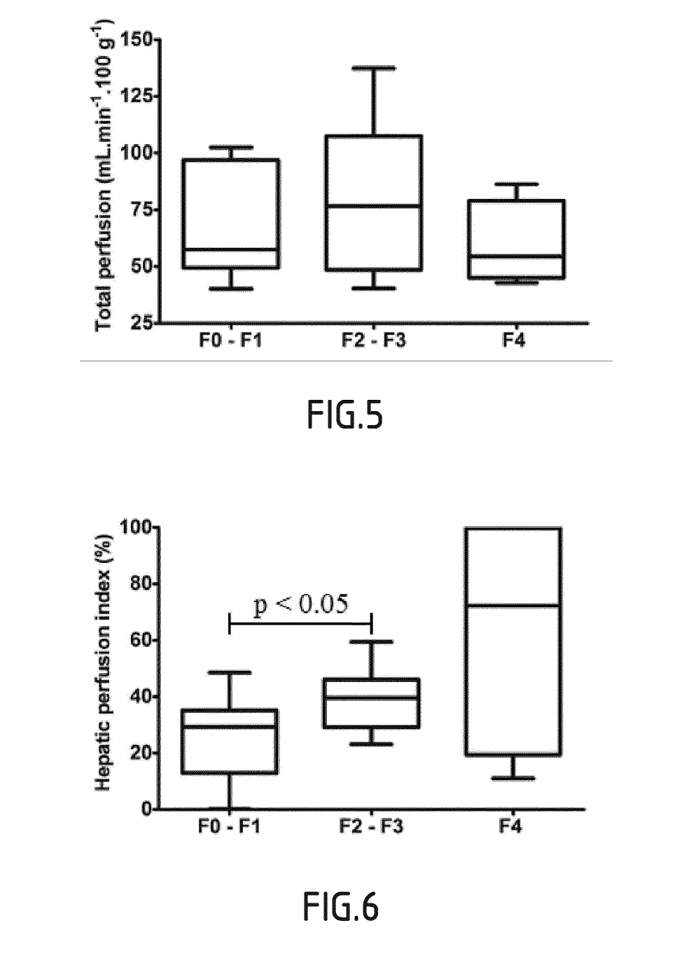

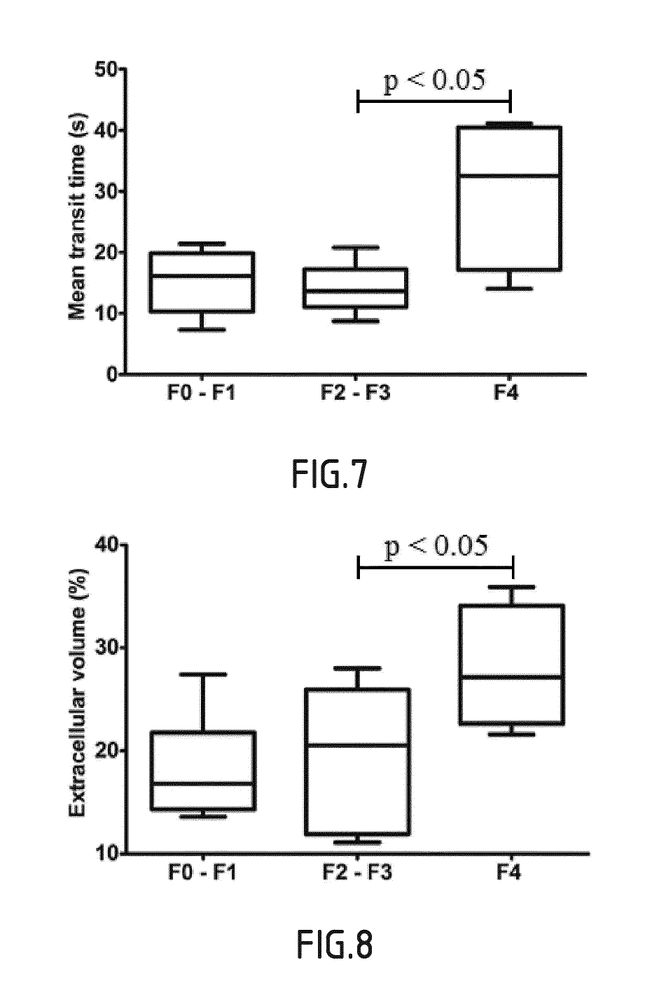

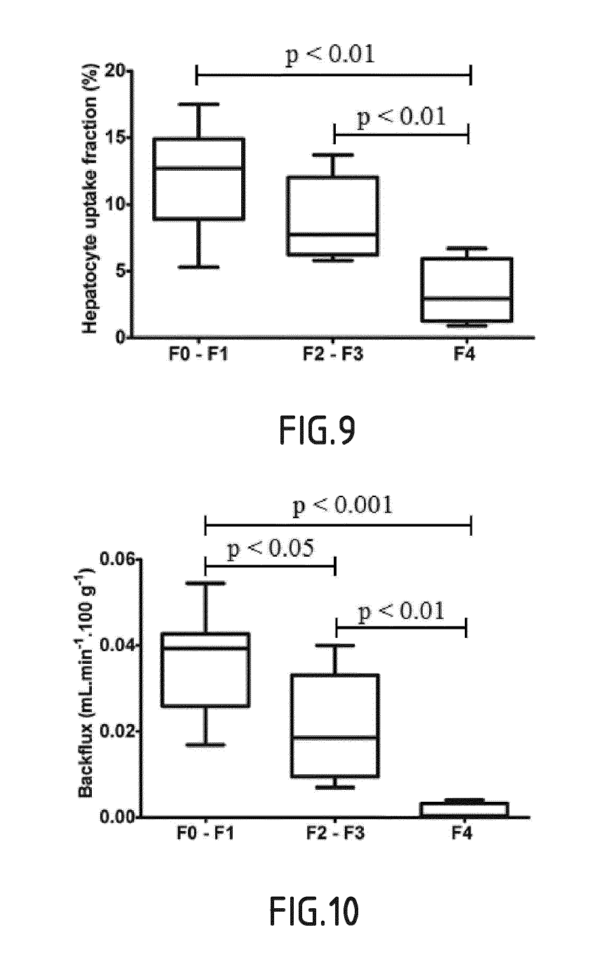

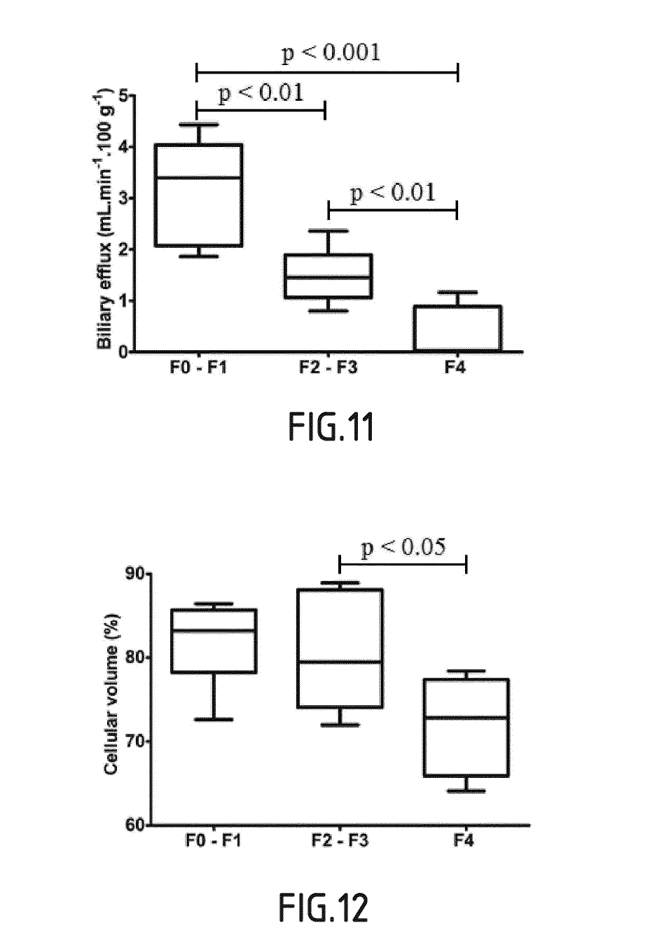

In the experience described in reference with FIGS. 4 to 17, the subjects are human beings.

The images are acquired with a magnetic resonance imaging technique.

The magnetic resonance imaging technique being enhanced by a contrast agent,

For instance, the contrast agent is gadoxetate.

More generally, all hepatospecific contrast media can be used as a contrast agent. For instance, Gd-BOPTA may be used.

The magnetic resonance imaging technique involving a dynamic contrast-enhanced acquisition.

According to the specific embodiment described, the dynamic contrast-enhanced acquisition is a gradient echo sequence.

In addition, the magnetic resonance imaging technique is carried out by a clinical system operating at magnetic field with a magnitude of 3.0 Tesla (T).

Each image associates to each pixel of the image the amplitude of the measured signal in the magnetic resonance imaging technique.

In other words, for each image, it can be defined a magnitude.

The method for post-processing images enables to obtain determined parameters which are detailed below.

The determined parameters are perfusion parameters and liver transport function parameters.

Perfusion parameters include the total perfusion which represents the contribution of arterial and portal perfusion, the hepatic perfusion index which represents the part or arterial perfusion over the total perfusion, the extracellular mean transit time which is the inverse of the venous transfer rate and the extracellular volume.

Liver transport function parameters include the hepatocyte uptake fraction, the sinusoidal backflux and the biliary efflux.

The method for post-processing images comprises three phases P1, P2 and P3, which are a phase P1 of extracting, a phase P2 of converting and a phase P3 of calculating.

At the phase of extracting P1, a time-intensity curve for at least one pixel of the images is extracted.

According to the specific embodiment described, from dynamic frames a signal intensity over the time S(t) is extracted pixel by pixel.

At the end of the extracting phase P1, for each pixel, the signal intensity over the time S(t) is known.

At the phase of converting P2, the extracted signal is converted into in a concentration signal.

By definition, a concentration signal is a signal representative of the evolution of the contrast agent concentration with time.

The phase of converting P2 comprises a step of converting the concentration of contrast agent concentration in blood into the concentration of contrast agent concentration in plasma.

For this, the input functions may be normalized by one minus hematocrit, the hematocrit being set to 45%. Since gadoxetate does not diffuse into red blood and to take into account only plasmatic exchange between input function and extracellular compartment in the liver, input functions were normalized by one minus the hematocrit.

The phase of converting P2 further comprises a step of interpolating the concentration signal.

The interpolation is, for instance, achieved by using spline curves.

At the phase of calculating P3, the at least one perfusion parameter and the at least one transport parameter are calculated.

The phase of calculating P3 enables to calculate the at least one perfusion parameter and the at least one transport parameter by using a fitting procedure applied on a model.

The model is a function which associates to a plurality of parameters each concentration signal.

The plurality of parameters are parameters which characterizes the kinetics of the elimination of the contrast agent by the liver, the liver being represented as a three-compartment organ with an extracellular compartment, a hepatocyte compartment and the intra-hepatic bile ducts.

The plurality of parameters comprises at least one perfusion parameter and at least one transport parameter.

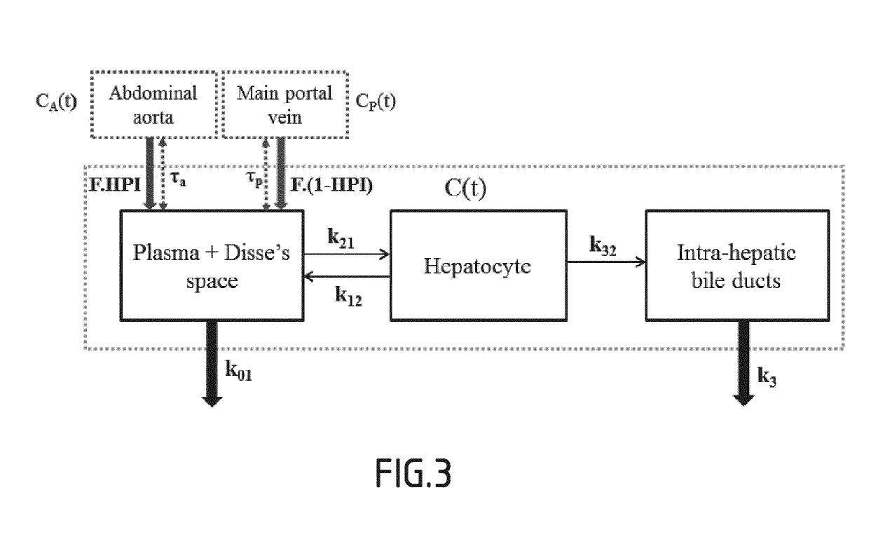

The model is thus a pharmacokinetic model modeling the kinetic of elimination of the tracer and is schematically illustrated by FIG. 3.

According to such model, the liver is a dual input three compartments. The three compartments are the extracellular compartment, the hepatocyte compartment and the intrahepatic bile ducts. The extracellular compartment comprises the intravascular compartment and the Disse space's. The hepatocyte compartment is also named the cellular compartment.

The extracellular compartment is the first compartment, the hepatocyte compartment is the second compartment and the intrahepatic bile ducts is the third compartment.

In normal operating, the gadoxetate first enters in the extracellular compartment by arterial and portal inputs according arterial and portal perfusion. A fraction of gadoxetate uptake into the hepatocyte where it can be excreted into the intrahepatic bile duct or redistributed into the extracellular compartment by sinusoidal backflux.

The non-uptake fraction of tracer directly washes out the liver through the hepatic veins to be next redistributed according to a venous transfer rate.

The previous exchanges are represented by a set of parameters which are listed below:
C(t) is the concentration of the tracer in the plasma. More precisely, C(t) is the concentration in the liver (more precisely in the region of interest). Concentration in the liver includes concentration in extracellular compartment (plasma+Disse space), in the hepatocyte, and in the intrahepatic bile duct.
F is the blood flow also named total perfusion,
C_{A}(t) is the arterial input function,
C_{P}(t) is the portal input function,
HPI is the hepatic perfusion index expressing the part of arterial perfusion over total perfusion F,
τ_{A} is the arterial delay corresponding to the temporal offset between the true input in the liver and measured input from arterial perfusion,
τ_{P} is the portal delay corresponding to the temporal offset between the true input in the liver and measured input from portal perfusion,
k₀₁ is the venous transfer rate,
k₂₁ denotes the hepatocyte uptake,
k₁₂ denotes the sinusoidal backflux,
k₃₂ denotes the portion transferred from the hepatocyte compartment to the intrahepatic bile ducts,
k₃ denotes the biliary efflux transfer rate,
c is a coefficient representing the difference of volume between the hepatocyte and the intrahepatic ducts, and
ρ is tissue mass volume.

Among the previous mentioned parameters, F, HPI, k01 are perfusion parameters. K21, k12 and k3 are hepatic transport function parameters.

A full demonstration of the expression of the model can be found in the experimental section.

According to this example, the model is the following function:

$$C ( t ) = F [ {C}_{A} ( t - {τ}_{A} ) HPI + {C}_{P} ( t - {τ}_{P} ) ( 1 - HPI ) ] ⊗ [ {e}^{- {k}_{01}} + ( {k}_{21} - \frac{{k}_{21} {k}_{3}}{{k}_{12}} {e}^{- ( {k}_{3} + {k}_{12} ) t} + c \cdot \frac{{k}_{21} {k}_{3}}{{k}_{12}} {e}^{- {k}_{3} t} ) ] ρ$$

The phase of calculating P3 comprises, according to the example of FIG. 2, a step of obtaining S50 the arterial and portal delays, a first step S60 of fitting procedure, a second step S70 of fitting procedure and a third step S80 of calculating.

At the step of obtaining S50, the arterial delay is calculated by using the FIG. 4 which represents the arterial input function (curve C1) portal input function (curve C2) and tissue response (other curve) recorded after retrospective respiratory motion correction. These curve well illustrated the absence of saturation effect with an arterial to tissular peak ratio=10 and an arterial to portal peak ratio=2. These curves also show the absence of important temporal noise linked to misregistrations between dynamic frames.

For these reasons, the arterial delay is measured as the temporal difference between the beginning of the arterial input and tissue response increases and portal delay is set to zero.

Arterial input function is extracted from a region of interest drawn by a user in the abdominal aorta.

Portal input function is extracted from a region of interest drawn by a user in the main portal vein.

Tissue response is extracted from a region of interest drawn by a user in the liver parenchyma.

At the first step S60, several parameters of the model are set to zero. More precisely, the biliary efflux and the sinusoidal backflux are neglected such that the model becomes a simplified model M1.

The simplified model M1 can be written as:

$$C ( t ) = F [ {C}_{A} ( t - {τ}_{A} ) HPI + {C}_{P} ( t - {τ}_{P} ) ( 1 - HPI ) ] ⊗ ( {e}^{- {k}_{01} t} + \frac{{k}_{21}}{{k}_{21} + F} ( 1 - {e}^{- {k}_{01} t} ) )$$

Tissue mass volume is accounted by multiplying by the tissue mass volume (equal to 1) such as described previously.

The simplified model M1 is a dual input bi compartment uptake model. The simplified model corresponds to the liver being represented as a two-compartment organ with an extracellular compartment and a hepatocyte compartment only.

By using a fitting technique which is a non-linear least-square fitting technique using pseudo-random initial conditions, determined parameters with a determined value are obtained.

In the example of FIG. 2, the perfusion parameters F, HPI and k₀₁ and the hepatocyte uptake k₂₁ are obtained.

During the second step S70, several parameters are set the determined value so that the model corresponds to a second model M2 in which the perfusion parameters F and HPI and the hepatocyte uptake k₂₁ are known, that is set their determined value at the first step. The second model M2 is written as follows:

$$C ( t ) = F [ {C}_{A} ( t - {τ}_{A} ) HPI + {C}_{P} ( t - {τ}_{P} ) ( 1 - HPI ) ] ⊗ [ {e}^{- {k}_{01}} + ( {k}_{21} - \frac{{k}_{21} {k}_{3}}{{k}_{12}} {e}^{- ( {k}_{3} + {k}_{12} ) t} + c \cdot \frac{{k}_{21} {k}_{3}}{{k}_{12}} {e}^{- {k}_{3} t} ) ] ρ$$

In this second model M2, the venous transfer rate k₀₁ is a free parameter, which means that the value of the first step is discarded without being used.

The fitting technique is again a non-linear least-square fitting technique using pseudo-random initial conditions

This enables to obtain the venous transfer rate k₀₁, the sinusoidal backflux k₁₂ and the biliary efflux transfer rate k₃.

In addition, according to the example of FIG. 2, the first step S60 is applied on images during a first interval of time and the second step S70 is applied on images during a second interval of time, the second interval of time including the first interval of time.

This means that the first interval of time comprises times which are each times of the second interval of time.

For instance, the ratio of the first interval of time to the second interval of time is inferior to 25%.

For instance, the first interval of time is comprises between 5 minutes to 10 minutes.

In the specific example of FIG. 2, the first interval of times comprises times which are inferior to 8 minutes while the second interval of times comprises times which are inferior to 38 minutes.

The calculating phase further comprises the step of calculating S80 at least one of the hepatocyte uptake fraction, the extracellular mean transit time and the extracellular volume based on the determined at least one perfusion parameter and the at least one transport parameter, the calculated parameter being one of determined parameter.

According to the example of FIG. 2, the three parameters that is the hepatocyte uptake fraction, the extracellular mean transit time and the extracellular volume are calculated based on the parameters F, k₀₁ and k₂₁.

For this, the hepatocyte uptake fraction E in % is calculated using the following equation:

$$E = \frac{{k}_{21}}{{k}_{21} + F}$$

At the same time, the extracellular mean transit time MTT in s is calculated using the following equation:

$$M T T = \frac{1}{{k}_{01}}$$

In addition, the extracellular volume Ve in % is calculated using the following equation:

$$Ve = \frac{F \cdot M T T}{1 - E}$$

By using the three previous equations, the hepatocyte uptake fraction, the extracellular mean transit time and the extracellular volume are determined at the calculating phase.

The method enables to access simultaneously at least one perfusion parameter and at least one transport parameter based on a single MRI acquisition during which the patient is authorized to breath.

In other words, the provided method can be achieved in only one experiment and provides the best accuracy in the prediction of the risk for a subject to suffer from a liver disease.

Such method may be used to study the treatment response in a liver disease.

This method can be used to study the treatment response in liver oncology.

This method can be used to study the treatment response in chronic liver diseases.

This method can be used to assess non-invasively the liver function before major liver resection to minimize the risk of post-operative liver failure, especially in patients with cirrhosis or even less advanced chronic diseases.

Finally, this method aims to provide with biomarkers of hepatic function and microperfusion which may be useful in liver oncology, in chronic liver disease or before major liver surgery. The method for post-processing may be used advantageously in other methods, the adaptation to these methods being immediate.

The embodiments and alternative embodiments considered here-above can be combined to generate further embodiments of the invention.

### Experimental Section

Experiments were carried out by an Applicant and are illustrated by the FIGS. 4 to 17. These experiments are detailed below.

### Abbreviations

The following abbreviations are used in the reminder of this section:
GRAPPA: generalized autocalibrating partial parallel acquisition;
MR: magnetic resonance;
ROC: receiver operating characteristic;
TWIST: time-resolved angiography with interleaved stochastic trajectories.

### Aim of the Study

Assessment of liver transport function is important to determine the prognosis of patients with chronic liver diseases, and is particularly required in patients with cirrhosis to determine the optimal timing for transplantation and determine whether or not transjugular intrahepatic portosystemic shunt insertion is contraindicated. Evaluation of total and regional liver function is also needed before major liver resection to minimize the risk of post-operative liver failure, especially in patients with cirrhosis or even less advanced chronic diseases such as steatosis, cholestasis, or chemotherapy toxicity.

During the eighties, imaging has been proposed to quantify the hepatic transport function such as scintigraphy following the injection of the hepatobiliary tracer t echnetium-99^{m} iminodiacetic acid. Despite the good sensitivity of scintigraphy, low spatial and temporal resolutions impede any methodological development. The pharmacokinetic properties of gadoxetate are similar to iminodiacetic acid family with a hepatocellular uptake and a biliary excretion through the OATP-B1 and MRP2 transporters respectively. In contrast to scintigraphy, the advantage of MR imaging is the ability to combine high spatial resolution, giving a morphological information, with a high temporal resolution, allowing to quantify the hepatic micro-perfusion in addition to transport function. Quantitative dynamic gadoxetate enhanced MR imaging was first published in anesthetized rabbits in 2004 and later applied to humans. In these studies, the hepatocyte gadoxetate uptake fraction and the input relative blood flow was estimated with a deconvolution method. By using a hybrid approach combining a deconvolution for plasma flow map calculation then pharmacokinetic modeling, it has been shown that it is possible to simultaneously measure hepatic micro-perfusion and hepatocyte gadoxetate uptake fraction. Nevertheless, whereas information about biliary excretion may provide a relevant clinical information, any approaches are able to simultaneously quantify micro-perfusion and the whole hepatic transport function (i.e the hepatocyte uptake and the biliary excretion). Thus, the aims of this study are to develop a method able to simultaneously quantify the hepatic micro-perfusion and the whole transport function with MR dynamic gadoxetate enhanced imaging. The clinical feasibility will be investigated in vivo, in patients with chronic liver diseases.

### Patients and Methods

### Subjects

The feasibility of the method was evaluated in a prospective study with liver biopsy as a gold standard on patients with liver chronic liver diseases. The institutional ethics committee approved the protocol and informed consent was obtained from each subject. Twenty-one consecutive patients (4 women, 17 men; mean age, 51.0±10.2 years) who were suspected of having chronic liver disease on clinical and biological data underwent in the same period liver biopsy and MR imaging. Mean delay between MR imaging and liver biopsy was 2.5 days (range: 0-20 days). All subjects were fasting during six hours before MR imaging. Exclusion criteria were the usual contra-indications for MR imaging.

### Histopathological Examination

Percutaneous liver biopsy was performed with a 1.4-mm-diameter needle (Hepafix; Braun, Melsungen, Germany). In 6 patients with ascites and/or trouble of blood crasis, liver biopsy was performed through a transjugular approach using a catheter needle with a diameter of 1.1 mm (Cook Bloomington, Ind., USA). The liver samples were fixed in buffered formalin and embedded in paraffin. Sections, 4 μm thick, were stained with hematoxylin-eosin and Masson trichrome and assessed by a pathologist blinded to the clinical data and to the results of MR imaging. The stage of fibrosis from F0 to F4 was evaluated semiquantitatively on Masson trichrome-stained slides according to the METAVIR staging system. For further analysis of fibrosis severity, a three-level staging of fibrosis was used (F0-F1: minimal fibrosis, F2-F3: intermediate fibrosis, and F4: cirrhosis). Moreover, patients with F≥2 were considered having significant fibrosis.

### MR Imaging

MR imaging was performed on a Siemens Skyra 3.0 T system (Siemens Medical Solutions, Erlangen, Germany). Dynamic acquisition was achieved with a three-dimensional time-resolved angiography with interleaved stochastic trajectories (TWIST) sequence employing the generalized autocalibrating partial parallel acquisition (GRAPPA). The signal was collected with the 32-channel phased array body and the spine coils.

The acquisition parameters were:
central region A, 40% sampling density;
peripheral region B, 50%, GRAPPA factor, 3×1 according to phase and slice direction;
echo time/repetition time/flip angle, 0.84 ms/2.44 ms/15°;
oversampling, 20% and 10% according to phase and slice direction; k-space partial filling, 6/8^{th}; and
745 Hz·pixel⁻¹ receiver bandwidth.

To minimize the flow-related enhancement in the aorta linked to slice entry phenomena, the acquisition plane was coronal with the following geometric parameters: field-of-view, 375×300×120 mm³; acquisition/reconstruction matrix, 140×160×24/200×160×40 with right/left phase encoding direction. TWIST temporal resolution was 2.1 s.

Gadoxetate (Primovist, Bayer, Berlin, Germany) was injected intravenously using an automatic injector (Medrad Spectris Solaris, Warrendale, PA) with an injection rate of 1 mL·s⁻¹ and flushed with physiologic saline (same volume and injection rate). The gadoxetate dose was 0.025 mmol·kg⁻¹.

Dynamic liver imaging was started simultaneously with the intravenous injection of gadoxetate and was performed in free breathing. Since gadoxetate kinetic decreases along the time after bolus injection (that is faster during perfusion phase than during uptake phase than during biliary excretion phases), the temporal resolution was artificially decreased over the time to minimize the total number of images.

To do this, pauses were added between dynamics as follow: for the 280 first dynamics: no pause, the effective temporal resolution was 2.1 s; for the next 150 dynamics: 5 s pause, the effective temporal resolution was 7.1 s, for the 60 last dynamics; 8 s pause, the effective temporal resolution was 10.1 s. Dynamic acquisition duration was close to 38 minutes.

### Image Reconstruction

### Model

To simultaneously quantify hepatic micro-perfusion and function transport parameters, a model was developed. In the liver, the quantity of gadoxetate Q between τ and τ+dτ can be expressed as the difference between incoming Qᵢₙ and outgoing gadoxetate quantities Qₒᵤₜ. This means that:

dQ=QᵢₙQₒᵤₜ

By considering the incoming blood flow equal to the outgoing blood flow, the previous equation becomes:

dQ=F(Cᵢₙ(τ)−Cₒᵤₜ(τ))dτ

Where:
Cᵢₙ is the concentration of gadoxetate into the incoming blood,
Cₒᵤₜ is the concentration of gadoxetate into the outgoing blood, and
F is the total perfusion.

Then, by integrating, it is obtained:

Q(t)=F∫₀^{t}(Cᵢₙ(τ)−Cₒᵤₜ(τ))dτ

By assuming the liver as a time-invariant, causal, linear and stationary dynamic system:

Cₒᵤₜ(t)=Cᵢₙ(t)⊗h(t)

Where:
h(t) is the impulse response of the system.
⊗ denotes the convolution product.

By manipulating the previous equations, it is obtained:

$$\begin{matrix}Q ( t ) = F {\int }_{0}^{t} ( {C}_{i n} ( τ ) - {C}_{i n} ( τ ) ⊗ h ( τ ) d τ \\ = F {\int }_{0}^{t} ( {C}_{i n} ( τ ) - ⊗ [ δ ( t ) - h ( τ ) ] ) d τ \\ = {FC}_{i n} ( t ) ⊗ R ( t )\end{matrix}$$

Where:
δ(t) is the delta function which is equal to 1 when t is superior or equal to 0,
R(t) is the residue function which represents the gadoxetate fraction still present in the liver along the time t. R(t) is equal to 1−∫₀^{t}h(τ)dτ.

Besides, liver having a double input (arterial and portal), the concentration of gadoxetate into the incoming blood can be expressed as:

Cᵢₙ(t)=C_{A}(t−τ_{A})HPI+C_{P}(t−τ_{P})(1−HPI)

Where
C_{A}(t) is the arterial input function,
C_{P}(t) is the portal input function,
HPI is the hepatic perfusion index expressing the part of arterial perfusion over total perfusion F,
τ_{A} is the arterial delay corresponding to the temporal offset between the true input in the liver and measured input from arterial perfusion, and
τ_{P} is the portal delay corresponding to the temporal offset between the true input in the liver and measured input from portal perfusion.

To express R(t) with kinetic indices kᵢⱼ, the liver is modeled with a model according to which the liver is a dual input three compartments. The three compartments are the extracellular compartment, the hepatocyte compartment and the intrahepatic bile ducts. The extracellular compartment comprises the intravascular compartment and the Disse space's.

In normal operating, the gadoxetate first enters in the extracellular compartment by arterial and portal inputs according arterial and portal perfusion. A fraction of gadoxetate uptake into the hepatocyte where it can be excreted into the intrahepatic bile duct or redistributed into the extracellular compartment by sinusoidal backflux.

The non-uptake fraction of tracer directly wash out the liver through the hepatic veins to be next redistributed.

Assuming that gadoxetate remains at tracer concentration, a linear kinetics is applicable to describe uptake and efflux of the gadoxetate by hepatocytes. This means that a linear system theory is applicable. Mathematically, such theory implies that

$$\frac{{dx}^{j} ( t )}{dt} = {A}_{ji} {x}^{i} ( t ) + {B}_{ji} {u}^{i} ( t )$$

xⁱ(t), j linear system theory, i=1 . . . n with n is the number of compartment, describe the time t evolution of gadoxetate quantity in each compartment.
Aⱼᵢ is the flow into and out of each compartment,
uⁱ(t) are the input control function for each compartment and
Bⱼᵢ the matrix describing the method of control application.

The residual function R(t) can be decomposed into two residue functions such as:

R(t)=Rₑ(t)+Rₚ(t)

Where:
Rₑ(t) represents the gadoxetate fraction still present into the extracellular compartment, and
Rₚ(t) represents the gadoxetate fraction still present into the hepatocyte compartment.

It is supposed that the gadoxetate fraction still present into the extracellular compartment Rₑ(t) can only be expressed as the following function of the venous transfer rate k₀₁:

Rₑ(t)=e^{-k}^{₀₁}^{t}

Using the linear system theory and taking into account that the adoxetate fraction still present into the hepatocyte compartment Rₚ(t) can be decomposed as the sum of the evolution of the gadotexate quantity into the hepatocyte compartment and the intrahepatic bile ducts. It can be obtained the following equation:

$${R}_{p} ( t ) = {k}_{21} - \frac{{k}_{21} {k}_{32}}{{k}_{32} + {k}_{12} - {k}_{3}} {e}^{- ( {k}_{32} + {k}_{12} ) t} + \frac{{k}_{21} {k}_{32}}{{k}_{32} + {k}_{12} - {k}_{3}} {e}^{- {k}_{3} t}$$

Where:
k₂₁ denotes the hepatocyte uptake,
k₁₂ denotes the sinusoidal backflux,
k₃₂ denotes the portion transferred from the hepatocyte compartment to the intrahepatic bile ducts, and
k₃ denotes the biliary efflux transfer rate.

By assuming k₃₂=k₃ and taking into account that at steady state, hepatocyte and intrahepatic distribution volumes are different, the gadoxetate fraction still present into the hepatocyte compartment Rₚ(t):

$${R}_{p} ( t ) = {k}_{21} - \frac{{k}_{21} {k}_{3}}{{k}_{12}} {e}^{- ( {k}_{3} + {k}_{12} ) t} + c \cdot \frac{{k}_{21} {k}_{3}}{{k}_{12}} {e}^{- {k}_{3} t}$$

With c is a coefficient representing the difference of volume between the hepatocyte and the intrahepatic ducts. Usually, c=0.01.

$$R ( t ) = {e}^{- {k}_{01}} + ( {k}_{21} - \frac{{k}_{21} {k}_{3}}{{k}_{12}} {e}^{- ( {k}_{3} + {k}_{12} ) t} + c \cdot \frac{{k}_{21} {k}_{3}}{{k}_{12}} {e}^{- {k}_{3} t} )$$

Therefore, the quantity of gadoxetate Q between τ and τ+dτ can be expressed as:

$$Q ( t ) = F [ {C}_{A} ( t - {τ}_{A} ) HPI + {C}_{P} ( t - {τ}_{P} ) ( 1 - HPI ) ] ⊗ [ {e}^{- {k}_{01}} + ( {k}_{21} - \frac{{k}_{21} {k}_{3}}{{k}_{12}} {e}^{- ( {k}_{3} + {k}_{12} ) t} + c \cdot \frac{{k}_{21} {k}_{3}}{{k}_{12}} {e}^{- {k}_{3} t} ) ] ρ$$

By multiplying by the tissue mass volume ρ, the previous equation can be written as follows:

$$C ( t ) = F [ {C}_{A} ( t - {τ}_{A} ) HPI + {C}_{P} ( t - {τ}_{P} ) ( 1 - HPI ) ] ⊗ [ {e}^{- {k}_{01}} + ( {k}_{21} - \frac{{k}_{21} {k}_{3}}{{k}_{12}} {e}^{- ( {k}_{3} + {k}_{12} ) t} + c \cdot \frac{{k}_{21} {k}_{3}}{{k}_{12}} {e}^{- {k}_{3} t} ) ] ρ$$

Therefore, all kinetic indices k as well as total perfusion F are expressed in volume by time units by mass unit. In this study, the conventional unity (that is. the mL·min⁻¹·100 g⁻¹) was used and ρ is considered equal to 1.

### Motion Correction

To compensate for misregistrations between dynamic frames, a retrospective respiratory motion correction algorithm was developed. First, for all frames of each 2D+t stack, a profile of intensity values along 10 lines integrated in the cranio-caudal direction and encompassing the hepatic dome was recorded. Next, to localize the lung-liver interface frame-by-frame, the maximum of the derivative was computed profile-by-profile. By comparison with a reference (chosen as the first frame), the offset, thus the rigid motion in the cranio-caudal direction over the time was quantified. Finally, the inverse motion was applied frame-by-frame by using a circular permutation.

### Quantification

From the motion corrected dynamic frames, arterial and portal input were recorded with freehand regions of interest (ROI) drawn in the abdominal aorta and the portal vein respectively. Tissue response was measured from a ROI placed in the right hepatic lobe anterior segment (segments V-VIII) and the right lobe posterior segment (segments VI-VII), two large ROIs covering an extended region without visible large vessels were drawn on three central slices. For inputs function and tissue response, signal intensity was converted into relative gadoxetate concentration as follows:

$$C ( t ) = \frac{S ( t ) - {S}_{0}}{{S}_{0}}$$

Where:
C(t) is the contrast agent concentration at time t,
S(t) the signal intensity at time t, and
S₀ the baseline signal intensity calculated by averaging the signal obtained on the non-contrast-enhanced dynamic scans.

To convert blood concentration into plasma concentration, the input functions were normalized by one minus hematocrit. A hematocrit of 45% was assumed in this study. To be able to include the delays (T_{A} and T_{P}) in the model (Eq. 23), the input functions were converted into a continuous temporal form instead of a discrete form by interpolation using spline curves.

The number of free parameters in the model being significant, and parameters being strongly connected between themselves, a dedicated data fitting algorithm was developed to avoid multiple local minima problem. In a first step, before the fitting procedure, the arterial delay was measured as the temporal difference between the beginning of arterial input and tissue response increases. The portal delay was fixed to zero.

Next, the data fitting procedure was stepwised as previously described. During the first step, the biliary efflux and sinusoidal backflux was neglected over early measurement (0 to 8 minutes) and perfusion parameters (F, HPI, k₀₁) and hepatocyte uptake (k₂₁) was computed with the following dual input bi compartment uptake model according to which:

$$C ( t ) = F [ {C}_{A} ( t - {τ}_{A} ) HPI + {C}_{P} ( t - {τ}_{P} ) ( 1 - HPI ) ] ⊗ ( {e}^{- {k}_{01} t} + \frac{{k}_{21}}{{k}_{21} + F} ( 1 - {e}^{- {k}_{01} t} ) )$$

During the second step, biliary efflux and backflux considered over all measurements (0 to 38 min) F, HPI and k₂₁ were fixed using the previous step and k₀₁ k₁₂ and k₃ were fitted.

Fitting procedure was performed with a constrained non-linear least square method using a multi-start trust-region reflective algorithm. For each optimization step, the fitting procedure was run with a grid of stochastic initial conditions generated within two bounds. Each fit procedure was carried out 50 times, with 50 different initializations.

Then, the hepatocyte uptake fraction E, the extracellular mean transit time MTT the extracellular volume Ve are calculated from the fitted parameters.

Statistical Analysis

The statistical significance of each MR quantitative parameter in discriminating between the patients according to fibrosis severity (minimal fibrosis, intermediate fibrosis and cirrhosis) was evaluated with the Kruskal-Wallis and Dunn's post-hoc tests. The Kruskal-Wallis and Dunn's post-hoc tests is a method for testing whether samples originate from the same distribution and used for comparing two or more independent samples of equal or different sample sizes. To assess their diagnostic value for the diagnosis of intermediate fibrosis (F≥2), receiver operating characteristic (ROC) analysis was performed. p-values<0.05 were considered to be statistically significant.

### Results

The cause of chronic liver disease was viral hepatitis B and/or C in eight patients, nonalcoholic steatohepatitis in seven, alcoholic hepatitis in three, auto-immune hepatitis, toxic hepatitis and Wilson's disease in one patient each. At histopathology, five patients were scored F0, four F1, four F2, four F3 and four F4.

Variations in perfusion and hepatic transport function parameters were observed between minimal fibrosis, intermediate fibrosis and cirrhosis.

This variation is notably illustrated by table 1 and FIG. 4.

The table 1 which illustrates the perfusion and hepatic transport function parameters according to fibrosis severity is reproduced below.

TABLE 1
Perfusion and hepatic transport function parameters
according to fibrosis severity

| | Minimal | Intermediate | |
|---|---|---|---|
| | fibrosis | fibrosis | Cirrhosis |
| | (F0-F1) (n = 9) | (F2-F3) (n = 8) | (F4) (n = 4) |
| F | 69.4 ± 24.6 | 79.4 ± 33.7 | 59.5 ± 18.8 |
| (mL · min⁻¹ · 100 g⁻¹) | | | |
| HPI (%) | 26.0 ± 15.6 | 39.0 ± 11.4 | 63.8 ± 43.6 |
| MTT(s) | 15.0 ± 5.0 | 14.1 ± 3.9 | 29.7 ± 9.7 |
| Ve (%) | 20.8 ± 6.2 | 21.8 ± 6.5 | 30.1 ± 12.5 |
| E (%) | 12.0 ± 3.8 | 8.7 ± 3.1 | 3.4 ± 2.5 |
| Sinusoidal | | | |
| backflux | 0.035 ± 0.012 | 0.021 ± 0.013 | 0.0012 ± 0.002 |
| (mL · min⁻¹ · 100 g⁻¹) | | | |
| Biliary efflux | 3.2 ± 1.0 | 1.5 ± 0.5 | 0.31 ± 0.57 |
| (mL · min⁻¹ · 100 g⁻¹) | | | |
| Cellular volume (%) | 81.9 ± 4.9 | 80.7 ± 6.8 | 72.1 ± 6.0 |

FIGS. 5 to 13 are boxplots of perfusion, and hepatic transport function parameters according to degree of liver fibrosis (minimal fibrosis, F0-F1), (intermediate fibrosis F2-F3) and cirrhosis (F4). Boxplots show the increase of arterial fraction in patients with intermediate fibrosis and cirrhosis, and the increase of mean transit time and extracellular volume in cirrhosis. Hepatocyte uptake fraction, sinusoidal backflux and biliary efflux decrease according to fibrosis severity. Cellular volume is reduced in cirrhosis. Lines within boxes represent median; lower and upper limits of boxes represent 25^{th} and 75^{th} percentiles and whiskers represent 10^{th} and 90^{th} percentiles.

By exploiting Table 1 and FIGS. 5 to 13, whereas total liver perfusion was similar (69.4±24.6 mL·min⁻¹·100 g⁻¹ versus 79.4±33.7 mL·min⁻¹·100 g⁻¹ versus 59.5±18.8 mL·min⁻¹·100 g⁻¹), the arterial fraction (given by the hepatic perfusion index) increased according to fibrosis severity (26.0±15.6%, 39.0±11.4% and 63.8±43.6% in minimal fibrosis, intermediate fibrosis and cirrhosis respectively; p<0.05 versus minimal and intermediate fibrosis). The extracellular mean transit time was similar between the fibrosis groups (15.0±5.0 s and 14.1±3.9 s in minimal and intermediate fibrosis respectively) and increased in cirrhosis (30.1±12.5 s, p<0.05 versus minimal and intermediate fibrosis). In the same way, the extracellular volume was similar between the fibrosis groups (18.1±4.6% and 19.3±6.8% for minimal and intermediate fibrosis respectively) and increased in cirrhosis (27.9±6.0%, p<0.01 and p<0.05 versus minimal and intermediate fibrosis).

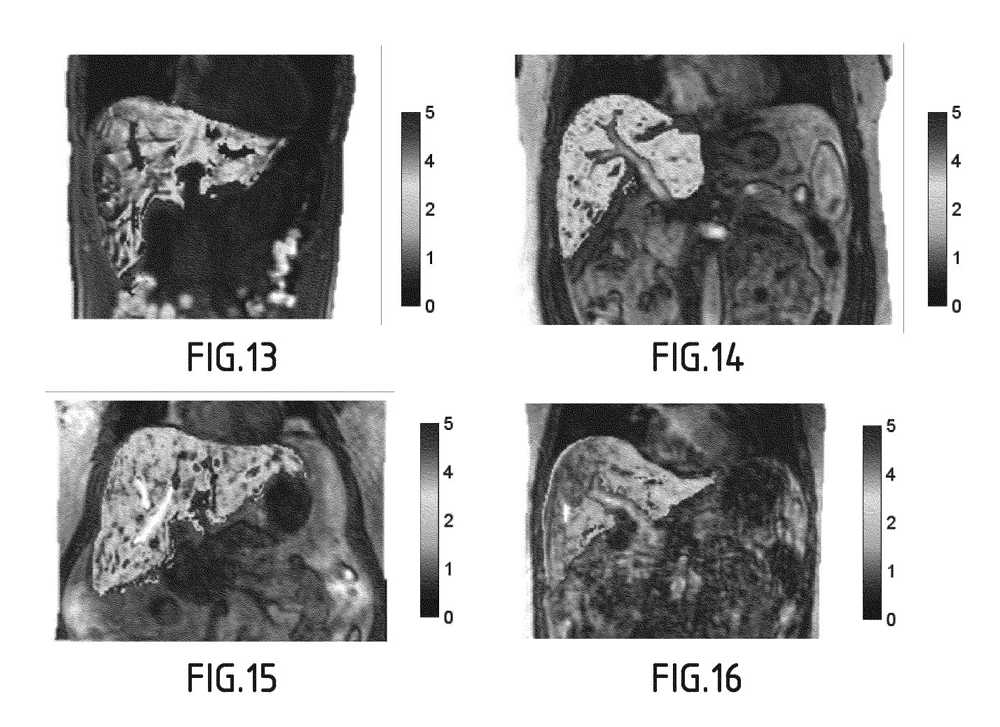

FIGS. 13 to 16 illustrate parametric maps of biliary efflux after pixel-by-pixel computation according to the fibrosis severity. FIG. 13 illustrates a map for a F0 patient, k₃ being equal to 3.53 mL·min⁻¹·100 g⁻¹; FIG. 14 illustrates a map for a F1 patient, k₃ being equal to 3.67 mL·min⁻¹·100 g⁻¹; FIG. 15 illustrates a map for a F2 patient, k₃ being equal to 2.23 mL·min⁻¹·100 g⁻¹ and FIG. 16 illustrates a map for a F3 patient, k₃ being equal to 1.64 mL·min⁻¹·100 g⁻¹

These maps well illustrated the decrease of biliary efflux according to fibrosis severity. The hepatocyte uptake fraction did not significantly decrease between the fibrosis (12.0±3.8% and 8.7±3.1% in minimal and intermediate fibrosis respectively, p=0.06), but significantly decreased in cirrhosis (3.4±2.5%, p<0.01 versus minimal and intermediate fibrosis). The biliary efflux decreased according to fibrosis severity (3.2±1.0 mL·min⁻¹·100 g⁻¹, 1.5±0.5 mL·min⁻¹·100 g⁻¹ and 0.31±0.57 mL·min⁻¹·100 g⁻¹ in minimal fibrosis, intermediate fibrosis and cirrhosis respectively, p<0.01 between each group). Similarly, the sinusoidal backflux decreased according to fibrosis severity (0.035±0.012 mL·min⁻¹·100 g⁻¹, 0.013±0.5 mL·min⁻¹·100 g⁻¹ and 0.0012±0.002 mL·min⁻¹·100 g⁻¹ in minimal fibrosis, intermediate fibrosis and cirrhosis respectively, p<0.01 between each group).

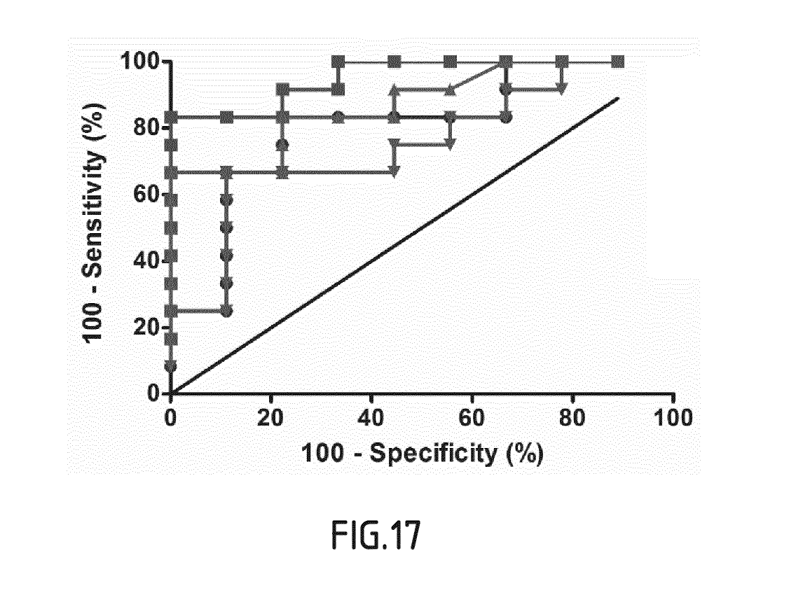

Based on the p-values, the hepatic perfusion index, the hepatocyte uptake fraction, the sinusoidal backflux, and the biliary efflux hepatobiliary enhancement were selected for ROC analysis. This selection is notably illustrated by table 2 and FIG. 17.

The table 2 illustrates the area under the receiver operating characteristic curve (AUROC), sensitivity, specificity, predictive positive value (PPV), negative predictive value (NPV) and accuracy (ACC) of hepatic perfusion index (HPI), hepatocyte uptake fraction, sinusoidal backflux and biliary efflux for diagnosing the significant fibrosis (F≥2). In other words, to diagnose a significant fibrosis (F≥2), AUROC (sensitivity/specificity) were 0.95 (83.3%/100%), 0.88 (83.3%/77.8%), 0.81 (83.3%/77.8%) and 0.75 (66.7%/88.9%) for the biliary efflux, sinusoidal backflux, hepatocyte uptake fraction and hepatic perfusion index. Whole ROC analysis results are summarized in Table. 2 which is reproduced below.

TABLE 2
ROC analysis

| | | | Sinusoidal backflux | Biliary efflux |
|---|---|---|---|---|
| | | Uptake | (mL · min⁻¹ · 100 | (mL · min−1 · 100 |
| | HPI | fraction | g⁻¹) | g−1) |
| Cut-off | >37.6% | <9.2% | <0.028 | <1.7 |
| AUROC | 0.75 | 0.81 | 0.88 | 0.95 |
| Sensitivity | 66.7 | 83.3 | 83.3 | 83.3 |
| (%) | | | | |
| Specificity | 88.9 | 77.8 | 77.8 | 100 |
| (%) | | | | |
| PPV (%) | 88.9 | 83.3 | 84.6 | 100 |
| NPV (%) | 66.7 | 77.8 | 87.5 | 81.8 |
| Accuracy | 76.2 | 81.0 | 85.7 | 90.5 |
| (%) | | | | |

FIG. 17 corresponds to ROC curves illustrating the performances of the hepatic perfusion index, the sinusoidal backflux, the hepatocyte uptake fraction and the biliary efflux as perfusion and liver function parameter to assess the significant fibrosis. Function parameters gave better performances than the hepatic perfusion index. Biliary efflux gave the best AUROC (0.95 vs. 0.88, 0.81 and 0.75 for the sinusoidal backflux, the hepatocyte uptake fraction and the hepatic perfusion index).

### Discussion

The previous results suggest the feasibility of simultaneous quantification of hepatic micro-perfusion and transport function with MR dynamic gadoxetate enhanced imaging and, to the best of our knowledge, we are the first to report a method able to do that. In this study, an increase of arterial perfusion fraction with fibrosis severity was observed, particularly in cirrhosis and a lengthening of gadoxetate extracellular mean transit time in cirrhosis. These variations of micro-perfusion according to the disease are consistent with previous results. Gadoxetate hepatocyte uptake fraction, biliary efflux and sinusoidal backflux significantly decreased according to fibrosis severity suggesting an alteration of the hepatic transport function according to the disease severity. In cirrhosis, the increase of extracellular distribution volume (or the decrease of cellular volume) linked to fibrotic material deposition in Disse space's can be an additional way explaining the drop of hepatocyte uptake fraction, biliary efflux and sinusoidal backflux. These results were is in agreement with previous results who showed a decrease of the relative enhancement twenty minutes after gadoxetate injection in advanced fibrosis and cirrhosis.

Besides, it appears that the hepatic function parameters were more relevant than micro perfusion parameters to diagnose significant fibrosis. Among hepatic function parameters, the biliary efflux was the most pertinent. This underlines the importance of complete hepatic liver function quantification. In this study, etiologies of disease were heterogeneous and this variability may explain the lower performance of the hepatic perfusion index to diagnose significant fibrosis in comparison with function parameters. Indeed, fibrosis localization varies according to the etiology (perisinusoidal in NASH and centrolobular in viral hepatitis) and thus differently affects liver perfusion as reflected by the large standard deviation for perfusion parameters in our groups.

In contrast to deconvolution based approaches, the presented method takes into account both arterial and portal inputs. Indeed, the obtained results shows that perfusion contribution from portal or arterial input over the total perfusion is drastically modified according to the disease.

Another advantage of the method is the absence of breath-holding requirement during dynamic acquisition since ghosting artifact was importantly reduced by the use of a key-hole acquisition with stochastic trajectories for k-space filling and misregistration between 2D+t frames were compensated by the retrospective respiratory motion correction including in our post-processing pipeline. In this regard, rather than to use a more conventional automatic registration algorithm we develop and include a dedicated algorithm in the reconstruction pipeline. The rationale behind this choice was that the functions of similarity used by automatic registration algorithms are sensitive to pixel intensity variation according to the time. Therefore, dynamic contrast enhancement confounds pixel intensity variations linked to the motion and induces substantial registration errors, particularly during the perfusion phase where signal intensity variations over the time are the most important. Amer-based semi-automatic methods could be an alternative to iconic, nevertheless, their use are limited by the prohibitive number of dynamics. Nevertheless, this method did not accounting for the non-rigid component of the motion and can be only used for coronal plane acquisitions.

To conclude, the simultaneous quantification of hepatic micro-perfusion and complete transport function is feasible with free breathing MR dynamic gadoxetate enhanced imaging. Hepatic transport function parameters may be useful to assess liver fibrosis in patients with chronic liver diseases.

1. A method for post-processing images of a region of interest in a subject to obtain determined parameters, the determined parameters comprising at least one perfusion parameter and at least one transport parameter, each perfusion parameter being relative to the hepatic perfusion and each transport parameter being relative to the hepatic function transport, the images being acquired with a magnetic resonance imaging technique, the magnetic resonance imaging technique being enhanced by a contrast agent, the magnetic resonance imaging technique involving successive echoes of a multiple-gradient echo sequence, each image associating to each pixel of the image the amplitude of the measured signal in the magnetic resonance imaging technique and the phase of the measured signal in the magnetic resonance imaging technique, the method for post-processing comprising at least the phase of:
extracting a time-intensity curve for at least one pixel of the images, to obtain at least one extracted signal,
converting the extracted signal in a concentration signal, a concentration signal being a signal representative of the evolution of the contrast agent concentration with time,
calculating the at least one perfusion parameter and the at least one transport parameter by using a fitting procedure applied on a model, the model being a function which associates to a plurality of parameters each concentration signal, the plurality of parameters being parameters which characterizes the kinetics of the elimination of the contrast agent by the liver, the liver being represented as a three-compartment organ with an extracellular compartment, a hepatocyte compartment and the intra-hepatic bile ducts, the plurality of parameters comprising at least one perfusion parameter and at least one transport parameter, the fitting procedure being applied in two steps:
a first step during which several parameters of the model are set to zero, the model becoming a simplified model corresponding to the liver being represented as a two-compartment organ with an extracellular compartment and a hepatocyte compartment only, to obtain determined parameters with a determined value and
a second step during which several parameters are set the determined value, to obtain the at least one perfusion parameter and the at least one transport parameter,
each step being achieved with a fitting technique being a non-linear least-square fitting technique using pseudo-random initial conditions.

2. A method for post-processing images according to claim 1, wherein the first step is applied on images during a first interval of time and the second step is applied on images during a second interval of time, the second interval of time including the first interval of time.

3. A method for post-processing images according to claim 2, wherein the ratio of the first interval of time to the second interval of time is inferior to 25%.

4. A method for post-processing images according to claim 2, wherein the first interval of time is comprises between 5 minutes to 10 minutes.

5. A method for post-processing images according to claim 1, wherein the phase of converting comprises a step of converting the concentration of contrast agent concentration in blood into the concentration of contrast agent concentration in plasma.

6. A method for post-processing images according to claim 1, wherein the phase of converting comprises a step of interpolating the concentration signal.

7. A method for post-processing images according to claim 1, wherein the plurality of parameters comprises the rate of exchanges between each of the three compartments.

8. A method for post-processing images according to claim 1, wherein the calculating phase comprises a step of calculating at least one of the hepatocyte uptake fraction, the extracellular mean transit time and the extracellular volume based on the determined at least one perfusion parameter and the at least one transport parameter, the calculated parameter being one of determined parameter.

9. A method for post-processing images according to claim 1, wherein the region of interest (ROI) includes a part of the liver.

10. A method for predicting that a subject is at risk of suffering from a liver disease, the method for predicting at least comprising the step of:
carrying out the steps of a method for post-processing images of the subject, to obtain determined parameters, the method for post-processing images being according to claim 1,
predicting that the subject is at risk of suffering from the liver disease based on the determined parameters.

11. A method for diagnosing a liver disease, the method for diagnosing at least comprising the step of:
carrying out the steps of a method for post-processing images of the subject, to obtain determined parameters, the method for post-processing images being according to claim 1, and
diagnosing the liver disease based on the determined parameters.

12. A method for identifying a therapeutic target for preventing and/or treating a liver disease, the method comprising the steps of:
carrying out the steps of a method for post-processing images of a first subject, to obtain first determined parameters, the method for post-processing images being according to claim 1 and the first subject being a subject suffering from the liver disease,
carrying out the steps of the method for post-processing images of a second subject, to obtain second determined parameters, the method for post-processing images being according to claim 1 and the second subject being a subject not suffering from the liver disease,
selecting a therapeutic target based on the comparison of the first and second determined parameters.

13. A method for identifying a biomarker, the biomarker being a diagnostic biomarker of a liver disease, a susceptibility biomarker of a liver disease, a prognostic biomarker of a liver disease or a predictive biomarker in response to the treatment of a liver disease, the method comprising the steps of:
carrying out the steps of a method for post-processing images of a first subject, to obtain first determined parameters, the method for post-processing images being according to claim 1 and the first subject being a subject suffering from the liver disease,
carrying out the steps of the method for post-processing images of a second subject, to obtain second determined parameters, the method for post-processing images being according to claim 1 and the second subject being a subject not suffering from the liver disease,
selecting a biomarker based on the comparison oldie first and second determined parameters.

14. A method for screening a compound useful as a probiotic, a prebiotic or a medicine, the compound having an effect on a known therapeutical target, for preventing and/or treating a liver disease, the method comprising the steps of
carrying out the steps of a method for post-processing images of a first subject, to obtain first determined parameters, the method for post-processing images being according to claim 1 and the first subject being a subject suffering from the liver disease and having received the compound,
carrying out the steps of the method for post-processing images of a second subject, to obtain second determined parameters, the method for post-processing images being according to claim 1 and the second subject being a subject suffering from the liver disease and not having received the compound,
selecting a compound based on the comparison of the first and second determined parameters.

15. A computer program product comprising instructions for carrying out the steps of a method according to claim 1 when said computer program product is executed on a suitable computer device.

16. A computer readable medium having encoded thereon a computer program according to claim 15.

17. A method for obtaining determined parameters by imaging a region of interest in a subject, the determined parameters comprising at least one perfusion parameter and at least one transport parameter, each perfusion parameter being relative to the hepatic perfusion and each transport parameter being relative to the hepatic function transport, the method comprising the steps of:
injecting a contrast agent,
acquiring images with a magnetic resonance imaging technique, the magnetic resonance imaging technique being enhanced by the contrast agent, the magnetic resonance imaging technique involving successive echoes of a multiple-gradient echo sequence, each image associating to each pixel of the image the amplitude of the measured signal in the magnetic resonance imaging technique and the phase of the measured signal in the magnetic resonance imaging technique,
the method for obtaining further comprising at least the phase of:
extracting a time-intensity curve for at least one pixel of the images, to obtain at least one extracted signal,
converting the extracted signal in a concentration signal, a concentration signal being a signal representative of the evolution of the contrast agent concentration with time,
calculating the at least one perfusion parameter and the at least one transport parameter by using a fitting procedure applied on a model, the model being a function which associates to a plurality of parameters each concentration signal, the plurality of parameters being parameters which characterizes the kinetics of the elimination of the contrast agent by the liver, the liver being represented as a three-compartment organ with an extracellular compartment, a hepatocyte compartment and the intra-hepatic bile ducts, the plurality of parameters comprising at least one perfusion parameter and at least one transport parameter, the fitting procedure being applied in two steps:
a first step during which several parameters of the model are set to zero, the model becoming a simplified model corresponding to the liver being represented as a two-compartment organ with an extracellular compartment and a hepatocyte compartment only, to obtain determined parameters with a determined value and
a second step during which several parameters are set the determined value, to obtain the at least one perfusion parameter and the at least one transport parameter,
each step being achieved with a fitting technique being a non-linear least-square fitting technique using pseudo-random initial conditions.
